(19) United States
(12) Patent Application Publication
(10) Pub. No.: US 2012/0232266 A1
(43) Pub. Date: Sep. 13, 2012

(54) REDUCTION OF ORGANIC COMPOUNDS WITH LOW AMOUNTS OF HYDROGEN

(75) Inventor: Martin Decristoforo, Kundl (AT)

(73) Assignee: SANDOZ AG, Basel (CH)

(21) Appl. No.: 13/501,253

(22) PCT Filed: Nov. 26, 2010
(86) PCT No.: PCT/EP2010/007193

§ 371 Date: Apr. 11, 2012

(30) Foreign Application Priority Data
Nov. 26, 2009 (EP) 09177215.2

(51) Int. Cl. C07D 265/32; C07C 5/11; C07C 237/48

(52) U.S. Cl. 544/177; 552/205; 585/268

(57) ABSTRACT

The present invention relates to a process for the reaction of a compound with hydrogen wherein the reaction is conducted using a hydrogen-containing gas comprising up to about 10 vol. % hydrogen and at least about 90 vol. % of an inert gas and wherein the compound to be reacted with hydrogen is provided in a liquid phase. The process of the present invention is particularly suitable for hydrogenation and hydrogenolysis reactions.

### FIELD OF THE INVENTION

The present invention relates to a process for the reaction of a compound with hydrogen wherein the reaction is conducted using a hydrogen-containing gas comprising up to about 10 vol. % hydrogen and at least about 90 vol. % of an inert gas and wherein the compound to be reacted with hydrogen is provided in a liquid phase. The process of the present invention is particularly suitable for hydrogenation and hydrogenolysis reactions.

### BACKGROUND OF THE INVENTION

The hydrogenation reactions are commonly employed in order to reduce compounds containing a double or triple bond. The sources of hydrogen vary depending on the type and scale of the reaction involved. While gaseous hydrogen in often used on an industrial scale, transfer hydrogenations using hydrogen donors such as hydrazine can be used in special applications.

In hydrogenolysis reactions a compound containing a carbon-carbon or carbon-heteroatom single bond is reacted with hydrogen whereby the carbon-carbon or carbon-heteroatom single bond is cleaved. Hydrogenolysis is used on a large scale for desulfurization in petroleum refining. It is also used commercially among others to prepare alcohols from the corresponding esters or to remove protecting groups like benzylesters, p-nitrobenzylesters benzhydrylesters etc.

CN-A-1569783 describes a non-petroleum route process for preparing ethylene using a gas mixture of pure acetylene, hydrogen and nitrogen as the raw material gas, wherein the volume content of acetylene in the raw material reaction gas is 10 to 40%.

At present, the reactions using hydrogen gas are typically conducted with pure hydrogen gas. Because the employed gas is explosive or forms explosive mixtures together with air, strict safety measures have to be taken. These safety measures make the reactions with hydrogen complicated and costly.

It is an object of the present invention to provide an improved process which is more simple and/or less costly than previous processes. A further object of the present invention is to provide a process which can be applied in large scale applications. Yet another object of the present invention is to provide a process which does not require the usual strict safety measures, e.g. protective measures against combustion and/or explosion usually required for catalytic hydrogenation reactions.

### SUMMARY OF THE INVENTION

The present invention relates to a process for the reaction of a compound with hydrogen wherein the reaction is conducted using a hydrogen-containing gas comprising up to about 10 vol. % hydrogen and at least about 90 vol. % of an inert gas and wherein the compound to be reacted with hydrogen is provided in a liquid phase.

### BRIEF DESCRIPTION OF THE DRAWINGS

FIG. 1 shows the ¹H-NMR-spectrum of the product of Example 1.

FIG. 2 shows the ¹H-NMR-spectrum of the product of Example 2.

FIG. 3 shows the HPLC-chromatogram of the product of Example 3.

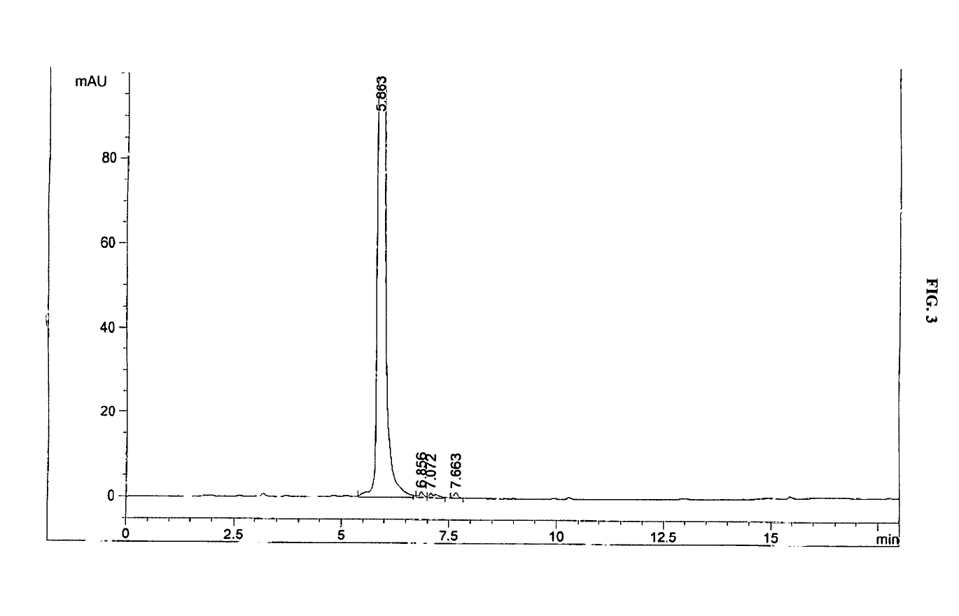

### DETAILED DESCRIPTION OF THE INVENTION

The present invention relates to a process for the reaction of a compound with hydrogen wherein the reaction is conducted using a hydrogen-containing gas comprising up to about 10 vol. % hydrogen and at least about 90 vol. % of an inert gas and wherein the compound to be reacted with hydrogen is provided in a liquid phase.

The present process can be applied to any process in which a compound can be reacted with a hydrogen-containing gas. Typical examples of such processes are hydrogenation reactions and hydrogenolysis reactions.

A hydrogenation reaction is defined as a reaction in which hydrogen (H₂) is reacted with a compound containing a double or triple bond and the hydrogen is added to the double or triple bond of the compound. In this reaction hydrogen is added without cleaving the linkage between the atoms connected by the double or triple bond. The resultant product corresponds to the initial compound but, depending on the employed hydrogenation reaction, has a single or double bond. The term “hydrogenation reaction” refers to the above mentioned reaction and, unless stated otherwise, does not include the step in which a catalyst is regenerated.

A hydrogenation reaction is shown schematically in the following scheme whereby atoms are denoted by ★:

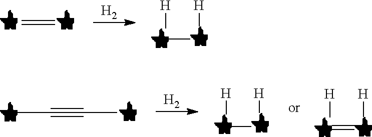

Many types of hydrogenation reactions are known in the art. The process of the present invention can be applied to all known hydrogenation reactions in which hydrogen gas is employed as the hydrogen source. A review over possible hydrogenation reactions which can be used in the present invention can be found in “Advanced Organic Chemistry•Part B: Reactions and Synthesis”, Chapter 5, 5^{th} edition, Francis A. Carey, Richard J. Sundberg, Springer Verlag, 2007, and M. Freifelder, “Catalytic hydrogenation in Organic Synthesis Procedures and Commentary, Wiley-Interscience, New York, 1978, which are incorporated by reference in their entirety.

A hydrogenolysis reaction is defined as a reaction in which a compound containing a carbon-carbon or carbon-heteroatom single bond is reacted with hydrogen whereby the carbon-carbon or carbon-heteroatom single bond is cleaved.

A hydrogenolysis reaction is shown schematically in the following scheme whereby atoms are denoted by ★:

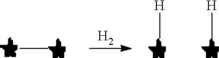

Many types of hydrogenolysis reactions are known in the art. The process of the present invention can be applied to all known hydrogenolysis reactions in which hydrogen gas is employed as the hydrogen source. A review over possible hydrogenolysis reactions which can be used in the present invention can be found in “Advanced Organic Chemistry•Part B: Reactions and Synthesis”, Chapter 5, 5^{th} edition, Francis A. Carey, Richard J. Sundberg, Springer Verlag, 2007; and M. Freifelder: “Catalytic hydrogenation in Organic Synthesis: Procedures and Commentary”, Wiley-Interscience, New York, 1978, which are incorporated by reference in their entirety.

The reaction of the present invention is conducted in the liquid phase. If the compound to be reacted with hydrogen is liquid, the liquid phase can be or can comprise the compound per se. Alternatively, the liquid phase can comprise a solution, suspension or emulsion of the compound which is to be reacted with hydrogen.

The liquid phase can be selected from any liquid which is suitable for the specific reaction which is to be conducted. Examples of typical solvents which can be used in the liquid phase include polar solvents such as water, alcohols (such as C₁₋₄ alcohols), esters (such as ethyl acetate, which can be used under gentle conditions known in the art), ethers (such as dioxane or THF, which can be used under gentle conditions, such as room temperature and atmospheric pressure), alkanes (such as cyclohexane) and organic acids (such as acetic acid).

While high amounts of hydrogen are typically employed in gas phase reactions, it has been surprisingly found that the process of the present invention can be conducted with low amounts of hydrogen in the hydrogen-containing gas which is passed, e.g. bubbled, through the liquid reaction medium. Without wishing to be bound by theory, it is assumed that the hydrogen in the hydrogen-containing gas becomes sufficiently dissolved in the liquid reaction medium or, if a catalyst is employed, can sufficiently interact with the catalyst even if very low amounts of hydrogen are present in the hydrogen-containing reaction gas mixture.

The process of the present invention could be conducted without a catalyst. However, a catalyst is typically desirable because the reaction with hydrogen can proceed under much milder conditions. The catalyst, if present, is typically either a homogeneous or heterogeneous catalyst, preferably a heterogeneous catalyst.

Homogeneous catalysts are soluble in the reaction medium. Examples of possible homogeneous catalysts include soluble complexes of transition metals. Examples of suitable transition metals include platinum group metals (such as Pd, Pt, Ru, Ir and Rh) as well as iron, cobalt, and nickel. Particular examples of possible homogeneous catalysts can be found in “Advanced Organic Chemistry•Part B: Reactions and Synthesis”, Chapter 5, 5^{th} edition, Francis A. Carey, Richard J. Sundberg, Springer Verlag, 2007 and M. Freifelder: “Catalytic hydrogenation in Organic Synthesis: Procedures and Commentary”, Wiley-Interscience, New York, 1978, which are incorporated by reference in their entirety.

Heterogeneous catalysts are not soluble in the reaction medium. Examples of possible heterogeneous catalysts are solid transition metals or their compounds, typically in a finely divided form, or transition metals or their compounds disposed on a support. Examples of suitable transition metals include platinum group metals (such as Pd, Pt, Ru, Ir and Rh) as well as iron, cobalt, and nickel. Chromite catalysts are further examples of possible heterogeneous catalysts. Carbon, calcium carbonate, barium sulfate, alumina and silica can be given as examples of possible supports. Examples of possible heterogeneous catalysts include Raney nickel, chromite catalysts, as well as platinum group metals on a support (e.g., platinum group metal on carbon such as platinum or palladium on carbon) or a platinum group metal as sponge or as oxide e.g. platinum dioxide (Adams catalyst). Particular examples of possible heterogeneous catalysts can be found in “Advanced Organic Chemistry•Part B: Reactions and Synthesis”, Chapter 5, 5^{th} edition, Francis A. Carey, Richard J. Sundberg, Springer Verlag, 2007 and M. Freifelder: “Catalytic hydrogenation in Organic Synthesis: Procedures and Commentary”, Wiley-Interscience, New York, 1978, which are incorporated by reference in their entirety.

The reaction can be conducted at any suitable pressure. The pressure will depend on the specific reaction which is to be conducted. Typically the reaction will be conducted at atmospheric pressure or elevated pressure. The pressure can, e.g., range from about 1×10⁵ Pa to about 3.5×10⁷ Pa. In one embodiment the pressure is about atmospheric pressure (about 1×10⁵ Pa). In another embodiment the pressure is about 1×10⁵ Pa to about 7×10⁵ Pa. In a further embodiment the pressure is about 7×10⁵ Pa to about 3.5×10⁷ Pa. The above values for gas pressure relate to the total pressure of the gas to be used in the hydrogenation reaction, not to the partial hydrogen pressure.

The reaction can be conducted at any suitable temperature. The temperature will depend on the specific reaction which is to be conducted. Typically the reaction will be conducted at room temperature (e.g., about 20° C. to about 25° C.) or at elevated temperature. The temperature can, e.g., range from about −25° C. to about 300° C., depending on the specific reaction to be conducted. In one embodiment the temperature is preferably from about −25° C. to about 250° C., alternatively from about −25° C. to about 100° C. and more preferably from about 0° C. to about 50° C.

Known additives and auxiliaries can be employed in the process of the present invention, as occasion requires. Examples are desactivating substances to influence the reactivity of the catalyst, for example lead as used for palladium on calcium carbonate catalysts, e.g. as detailed in Lindlar, H.; Dubuis, R. (1973), “Palladium Catalyst for Partial Reduction of Acetylenes”, Org. Synth., Coll. Vol. 5: 880. Catalysts with modified reactivity are, for example, employed for the partial reduction of carbon-carbon triple bonds to carbon-carbon double bonds and for the reduction of acid chlorides to aldehydes.

The process of the present invention can be conducted in a batch or continuous manner. In a preferred embodiment, it is conducted by continuously flowing the hydrogen-containing gas through the liquid phase. In a preferred embodiment, the gas is simply bubbled through the reaction liquid. Alternatively, the gas can be injected by means of a jet or by means of a sintered metal or glass candle. The gas also can be superimposed over the liquid in an autoclave at elevated pressure, in this case it is to be changed several times until the reaction is finished.

The hydrogen-containing gas comprises up to about 10 vol. % hydrogen and at least about 90 vol. % of an inert gas. A skilled person will be able to determine the lower limit of hydrogen which is suitable for the reaction which is to be conducted by way of a simple series of experiments. For instance, he could start with an initial amount of 5 vol. % hydrogen and reduce the amount of hydrogen in the hydrogen-containing gas in a stepwise manner and observe, whether the desired product resulting from hydrogenation or hydrogenolysis still forms.

Surprisingly, the present inventors have discovered that the overall reaction conditions for the process of the invention remain essentially the same with regard to temperature and pressure as compared to the corresponding process which uses pure hydrogen. This means that a compound which can be reacted with hydrogen in a liquid reaction medium at room temperature and under ambient pressure using pure hydrogen as the reaction gas can also be reacted in the same liquid reaction medium at room temperature and under ambient pressure using the reaction gas mixtures used in the process of the present invention. Thus, the skilled person can start from the ample knowledge about reactions with hydrogen which employ pure hydrogen as a reaction gas and can use these conditions as a starting point by replacing a gas containing 100 vol. % hydrogen by the reaction gas mixtures used in the process of the present invention.

The process of the invention is preferably conducted using a gas comprising about 0.1 to about 10 vol. % hydrogen and about 90 to about 99.9 vol. % of an inert gas. In a preferred embodiment the gas comprises about 1 to about 7 vol. % hydrogen and about 93 to about 99 vol. % of an inert gas, more preferably the gas comprises about 2 to about 6 vol. % hydrogen and about 94 to about 98 vol. % of an inert gas, most preferably about 5 vol. % hydrogen and about 95 vol. % of an inert gas. The commercially available mixture which consists of about 5 vol. % hydrogen/95 vol. % nitrogen is particularly preferred in the process of the invention.

In a preferred embodiment the gas consists essentially of the above indicated amounts of hydrogen and the inert gas. In this context “consists essentially of” refers to a gas which can include up to about 5 vol. %, preferably up to about 2 vol. %, more preferably up to about 1 vol. %, components other than hydrogen and the inert gas. In a further preferred embodiment the gas consists of above indicated amounts of hydrogen and the inert gas.

The inert gas can be any gas which is inert in the reaction at issue. Examples of inert gases include nitrogen and noble gases (such as argon) as well as mixtures thereof. In view of its cost, nitrogen is the preferred inert gas.

By employing the above described hydrogen-containing gas, the present invention provides a simple, cost effective and safe method for conducting reactions with hydrogen. Because the gas is not explosive either alone or in combination with air, it is possible to avoid the strict safety measures which were previously required for reactions with pure hydrogen. This enables the skilled person to use equipment for reactions with hydrogen which would have previously been considered unsuitable for this purpose due to lack of sufficient safety measures and/or to work in environments which would have previously been considered unsuitable for this purpose due to lack of sufficient safety measures.

The substrate (i.e., the compound to be reacted with hydrogen) is not particularly limited and is any compound which is susceptible to the desired reaction, e.g. the desired hydrogenation or hydrogenolysis reaction. Preferably the compound is an organic compound, more preferably having a molecular weight from 28 Da to 100 kDa, even more preferably from 40 Da to 50 kDa, such as from 50 Da to 10 000 Da.

In the case of a hydrogenation reaction the substrate is a compound containing a double or triple bond.

The compound is typically an organic compound. In one embodiment the compound is non-polymeric. The double or triple bond is preferably selected from the group consisting of

### C═C C≡C NO₂

### C═N C≡N

### C═O

### and —N═N—

Examples of compounds including suitable double or triple bonds include alkenes, alkynes, ketones, aldehydes, nitro compounds, imines, oximes, nitriles, aryl compounds and heteroaryl compounds, hydrazones, azines and azo compounds, with alkenes, alkynes, ketones, aldehydes, esters, nitro compounds, imines, oximes and nitriles being preferred and alkenes, alkynes, nitro compounds, imines and oximes being even more preferred.

Typical hydrogenation reactions include the following:

(i) reduction of alkene moiety to an alkane moiety;
(ii) reduction of an alkyne moiety to an alkene moiety;
(iii) reduction of an alkyne moiety to an alkane moiety;
(iv) reduction of a nitro moiety to an amine moiety;
(v) reduction of an imine moiety to an amine moiety;
(vi) reduction of an oxime moiety to an amine moiety; and
(vii) reduction of a nitrile group to an amine group;
(viii) reduction of a ketone moiety to an alcohol moiety;
(ix) reduction of an aldehyde moiety to an alcohol moiety;
(x) reduction of an aromatic moiety to the corresponding saturated cyclic moiety;
(xi) reduction of a heteroaryl moiety to the corresponding saturated hetero ring moiety.
(xii) reduction of an acid chloride moiety to the corresponding aldehyde (Rosenmund reduction)

Reactions (i) to (ix) are more preferred, reactions (i) to (vi) are even more preferred. In general, less harsh conditions can be employed for the more preferred reactions. In particular, the most preferred reactions work even at room temperature and ambient pressure to a slightly elevated pressure of not more than 7*10⁵ Pa. Suitable catalysts and/or reaction conditions for a particular substrate to be reacted with hydrogen can be found in “Advanced Organic Chemistry•Part B: Reactions and Synthesis”, Chapter 5, 5^{th} edition, Francis A. Carey, Richard J. Sundberg, Springer Verlag, 2007 and M. Freifelder: “Catalytic hydrogenation in Organic Synthesis: Procedures and Commentary”, Wiley-Interscience, New York, 1978, which are incorporated by reference in their entirety.

One possible application of the embodiment in which an alkene moiety is reduced to an alkane moiety is the hydrogenation as applied during the preparation of dihydrocodeine from codeine or of dihydroergotalcaloides from ergotamine, ergocrystine, ergotoxine or paspalic acid. This hydrogenation is typically conducted using a heterogeneous catalyst such as a catalyst based on Pd, Pt, Ir or Ni and proceeds quickly even at RT and about atmospheric pressure (about 1×10⁵ Pa). For suitable conditions see also example 2.

One possible application of the embodiment in which a nitro moiety is reduced to an amine moiety is the hydrogenation of 9-nitrominocycline, for example during the preparation of tigecycline. This hydrogenation is typically conducted using a heterogeneous catalyst such as a catalyst based on Pd, Pt, Ir or Ni and proceeds quickly even at RT and about atmospheric pressure (about 1×10⁵ Pa). For suitable conditions see also Example 3.

One possible application of the embodiment in which a C═N moiety is reduced to an amine moiety is the hydrogenation of aprimin, for example, during the preparation of aprepitant. This hydrogenation is typically conducted using a heterogeneous catalyst such as a catalyst based on Pd, Pt, Ir or Ni.

Examples of possible homogeneous catalysts for hydrogenation reactions include Wilkinson's catalyst (Ph₃P)₃RhHal), Crabtree's catalyst ([(tris-cyclohexylphosphine) Ir (1,5-cyclooctadiene) (pyridine)] PF₆⁻) and Brown's catalyst ([(Ph₂P(CH₂)₄PPh₂)Rh (nbd)]⁺BF₄⁻). All of these catalysts can be employed in the present invention, for example, to hydrogenate alkenes.

If desired, the hydrogenation can be conducted in an enantioselective manner by using chiral catalysts. Examples of possible enantioselective catalysts include transition metal complexes with DIOP, CHIRAPHOS, PROPHOS, PHENPHOS, CYCPHOS, DBPP, NORPHOS, CAMPHOS, DPCP, PYRPHOS, BPPM, PPPFA, DUPHOS, DIPHEMP, BINAP, DIPAMP, and DINAP.

A further example of a possible hydrogenation reaction is the reaction with a Lindlar catalyst.

The above mentioned catalysts are given as examples of possible catalysts for hydrogenation reactions which can be used in the present invention. However, they serve as an illustration and should not be construed as a limitation of the present invention, which is not restricted thereto.

An example of the hydrogenation of a nitro moiety is provided in the below reaction scheme

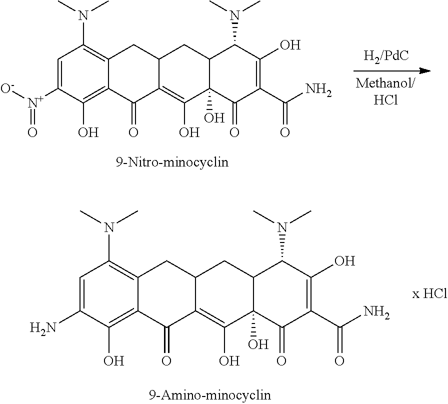

wherein the catalyst is 10% Palladium on charcoal, moistened with 50% of water and the process is carried out under the following conditions: A 3-5% solution of the substrate in methanol/hydrochloric acid is charged with an amount of catalyst corresponding to 15-20% w/w of the amount of substrate (on dry basis) and then a 5 v % hydrogen/95 v % nitrogen mixture is bubbled through the slurry at 20-25° C. and at a slight overpressure of approx. 100 mbar until the starting material has disappeared, as detected by HPLC.

An example of the hydrogenation of an olefin to a saturated hydrocarbon is provided in the below reaction scheme:

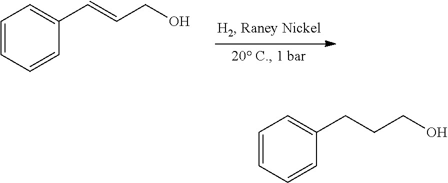

An example of the hydrogenation of an alkyn to a saturated hydrocarbon is provided in the below reaction scheme:

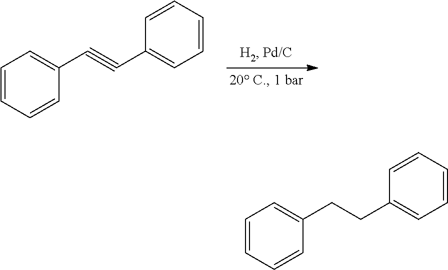

An example of the hydrogenation of a nitrile to a primary amine is provided in the below reaction scheme:

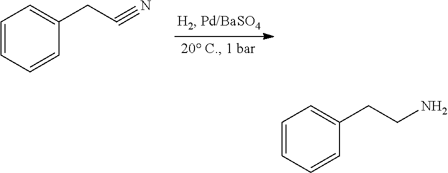

The skilled person will, however, appreciate that more typically the reduction of nitriles to primary amines requires elevated temperatures of between 50° C. and 100° C. and elevated pressure.

In a particularly preferred embodiment, the present invention relates to a process for the reaction of a compound with hydrogen, wherein the reaction is a hydrogenation reaction and is conducted using a hydrogen-containing gas comprising about 1 vol. % to about 7 vol. % hydrogen and about 93 vol. % to about 99 vol. % of an inert gas, wherein the compound to be reacted with hydrogen is provided in a liquid phase, wherein the compound is an organic compound having a molecular weight from 50 Da to 10 000 Da, wherein the pressure is about 1×10⁵ Pa to about 7×10⁵ Pa, wherein the temperature is from about 0° C. to about 50° C., in particular wherein the substrate for the hydrogenation reaction is a compound containing a double or triple bond which is susceptible to cleavage under the above conditions of temperature and gas pressure, in particular wherein the reaction is selected from the group consisting of the reduction of alkene moiety to an alkane moiety, reduction of an alkyne moiety to an alkene moiety, reduction of an alkyne moiety to an alkane moiety, the reduction of a nitro moiety to an amine moiety, the reduction of an imine moiety to an amine moiety, and the reduction of an oxime moiety to an amine moiety.

In the case of a hydrogenolysis reaction the substrate is a compound containing a carbon-carbon or carbon-heteroatom single bond which is susceptible to cleavage in a reaction with hydrogen. The compound is typically an organic compound. In one embodiment the compound is non-polymeric. The compound preferably has a moiety selected from the group consisting of

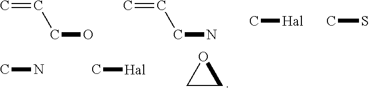

wherein the bond which is cleaved is indicated as a bold line.

Typical hydrogenolysis reactions include the following:

(i) removal of a benzyloxycarbonyl group by hydrogenolysis;
(ii) the reaction of a benzyl ester to a corresponding carboxylic acid and toluene;
(iii) the reaction of a benzyl ether to the corresponding benzyl compound and alcohol;
(iv) the reaction of a benzyldialkylamine to the corresponding dialkylamine and toluene;
(v) the reaction of a compound having a C-Hal bond to the corresponding compound having a C—H bond (wherein Hal is Cl, Br, I, or F; preferably I, Br or Cl; more preferably I or Br; even more preferably I);
(vi) the ring opening of an epoxide to the corresponding alcohol;
(vii) the cleavage of a C—S bond to result in a corresponding compound having a C—H bond and hydrogensulfide; and
(viii) the reaction of an ester to a corresponding primary alcohol.

Reactions (i) to (v) are preferred, reactions (i) to (iv) are more preferred and reactions (i) and (ii) are even more preferred.

In one preferred embodiment it is possible to employ the hydrogenolysis reaction according to the present invention to remove protecting groups. An example of this embodiment is the hydrogenolysis of an optionally substituted benzylether to an alcohol and the optionally substituted benzyl compound.

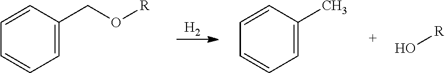

wherein the phenyl ring can be optionally substituted (e.g. by a methoxy or halogen) and wherein R is an residue compatible with the catalytic hydrogenation reaction under the particular conditions employed.

A further preferred example of a hydrogenolysis process of the invention for the removal of a protecting group is the cleavage of a benzyloxycarbonyl (Cbz) group.

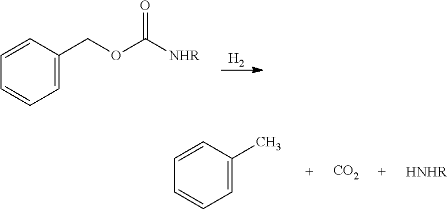

wherein the phenyl ring can be optionally substituted by a residue compatible with the catalytic hydrogenation reaction under the particular conditions employed (e.g. by alkyl, methoxy, halogen) and wherein R is an residue compatible with the catalytic hydrogenation reaction under the particular conditions employed. Cleavage of the benzyloxycarbonyl (Cbz) group is for example room temperature at about atmospheric pressure.

An example of another type of hydrogenolysis reaction includes the Rosenmund reduction, in which an acid chloride is reduced to the corresponding aldehyde with hydrogen in the presence of a partially desactivated palladium catalyst (desactivation with chinoline, sulfur compounds and the like).

In a particularly preferred embodiment, the present invention relates to a process for the reaction of a compound with hydrogen, wherein the reaction is a hydrogenolysis reaction and is conducted using a hydrogen-containing gas comprising about 1 vol. % to about 7 vol. % hydrogen and about 93 vol. % to about 99 vol. % of an inert gas, wherein the compound to be reacted with hydrogen is provided in a liquid phase, wherein the compound is an organic compound having a molecular weight from 50 Da to 10 000 Da, wherein the pressure is about 1×10⁵ Pa to about 7×10⁵ Pa, wherein the temperature is from about 0° C. to about 50° C., in particular wherein the substrate for the hydrogenolysis reaction is a compound containing a carbon-carbon or carbon-heteroatom single bond which is susceptible to cleavage under the above conditions of temperature and gas pressure, in particular wherein the reaction is the removal of a benzyloxycarbonyl group.

The present invention also relates to the use of a hydrogen-containing gas comprising up to about 10 vol. % hydrogen and at least about 90 vol. % of an inert gas for the catalytic hydrogenation or hydrogenolysis of an organic compound susceptible to catalytic hydrogenation or hydrogenolysis, wherein the substrate for catalytic hydrogenation or hydrogenolysis is provided in a liquid phase, in particular to the uses resulting from the application of the above described processes of the present invention.

The above mentioned catalysts are given as examples of possible catalysts for hydrogenolysis reactions which can be used in the present invention. However, the present invention is not restricted thereto.

The invention will now be explained with the help of the following examples. However, these examples should not be construed so as to be in any way limiting to the scope of the present invention.

### EXAMPLES

### Example 1

4.46 g diphenylacetylene were dissolved in 150 mL methanol. 1 g 10% palladium on carbon (available as RD-9210 from Hindustan Platinum Inc) was added. A mixture of 95 vol. % N₂ and 5 vol. % H₂ (available from Linde Gas) was passed through the suspension for 11 hours at a flow rate of approx. 30 L/h at room temperature (20-25° C.) and an overpressure of 100 mbar. The catalyst was removed by filtration. The solution was concentrated in vacuo. The resultant product was isolated by filtration and dried. 2.93 g diphenylethane were obtained.

¹H-NMR (CDCl₃, 300 MHz): 2.97 ppm (s), 4H, 2×CH₂; 7.21-7.26 ppm (m), 6H, 2×H3/4/5 arom.; 7.30-7.36 ppm (m), 4H, 2×H2/6 arom.

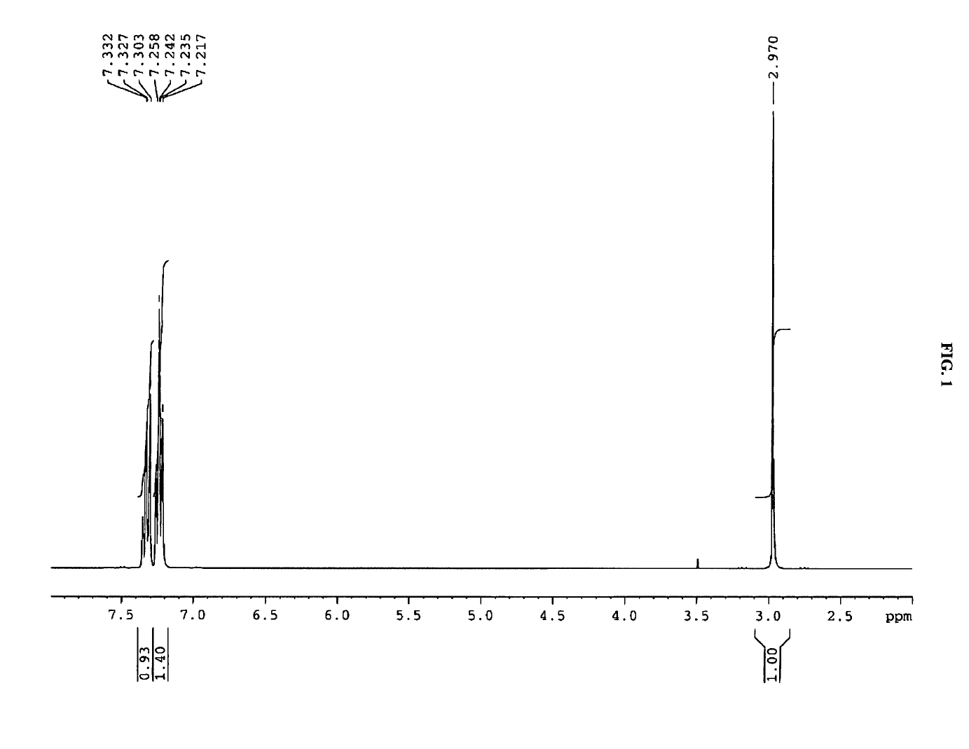

The NMR-spectrum of the product is shown in FIG. 1. Reduction of the alkyne to the alkane was essentially complete, with no detectable products from incomplete reduction of the triple bond to the alkene level, as can be taken from ratio of the integral for the alkane protons at 2.97 ppm to the sum of the integrals for the aromatic protons at around 7.2 to 7.3 ppm on the one hand and the absence of a peak corresponding to olefinic protons (between 5 ppm and 7 ppm) on the other.

### Example 2

0.94 g of 2-[(1R)-1-[3,5-bis(trifluoromethyl)phenyl]ethoxy]-3-(4-fluorophenyl)-5,6-dihydro-2H-1,4-oxazine were dissolved in 150 mL methanol. 1 g 10% palladium on carbon (available as RD-9210 from Hindustan Platinum Inc) was added. A mixture of 95 vol. % N₂ and 5 vol. % H₂ (available from Linde Gas) was passed through the suspension for 6.5 hours at a flow rate of 30 L/h. The temperature was 25 to 30° C. and the overpressure was approx. 150 mbar. The catalyst was removed by filtration. The solution was concentrated in vacuo until an oil was obtained. 0.9 g 2-[(1R)-1-[3,5-bis(trifluoromethyl)phenyl]ethoxy]-3-(4-fluorophenyl)-(2R,3S)-morpholine were obtained.

¹H-NMR (DMSO-d6, 300 MHz): 1.36 ppm (d, 3H, J=6.6 Hz) CH₃, 2.97 ppm (m, 2H) CH₂; 3.50 ppm (d, 1H, J=10.2 Hz) ½ CH₂; 3.92 ppm (d, 1H, J=2.4 Hz) CH; 3.99 ppm (m, 1H) ½ CH₂; 4.41 ppm (d, 1H, J=2.4 Hz) CH; 4.69 ppm (q, 1H, J=6.6 Hz) CH; 7.05 ppm (t, 2H, J=9.0 Hz) 2×CH; 7.33 ppm (dd, 2H, J¹=2.1 Hz, J²=5.7 Hz) 2×CH; 7.40 ppm (s, 2H) 2×CH, 7.85 ppm (s, 1H) CH.

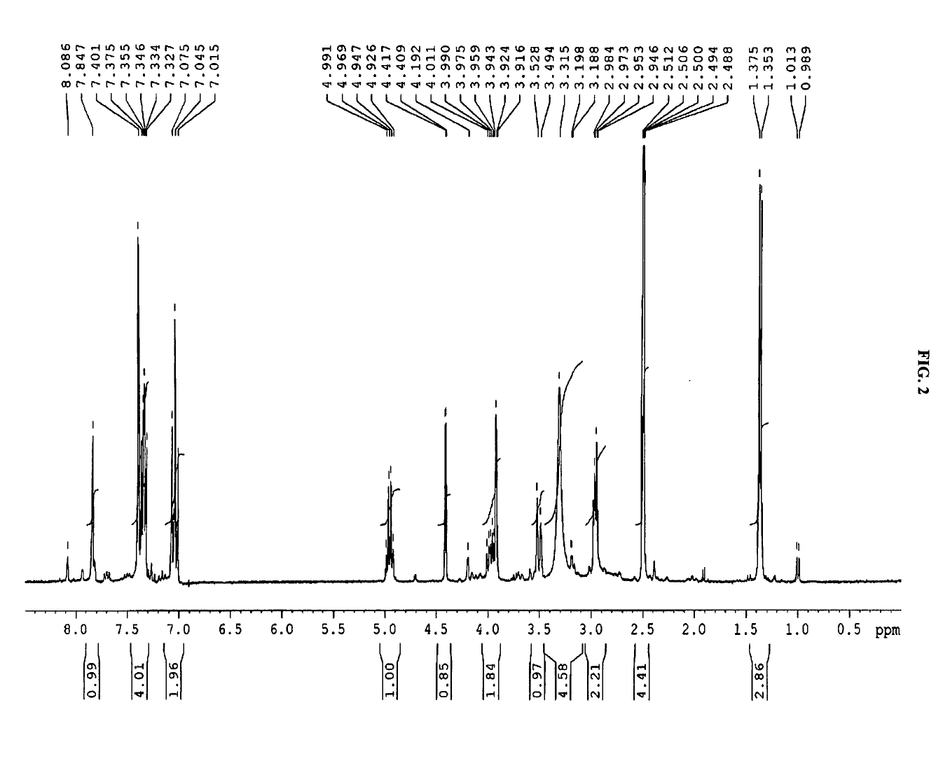

The NMR-spectrum of the product is shown in FIG. 2. The integrals and the type of coupling of the signals at 3.9 and 4.4 ppm are characteristic for the hydrogenation product (protons in the morpholino ring). The absence of a signal at 5.15 ppm (characteristic for the starting material) indicates the essential completeness of the reaction.

### Example 3

88.8 g (4S,4aS,5aR,12aS)-4,7-bis(dimethylamino)-9-nitro-1,4,4a,5,5a,6,11,12a-octahydro-3,10,12,12a-tetrahydroxy-1,11-dioxo-2-naphthacencarboxamide were dissolved in 2.7 L methanol and 40 mL concentrated hydrochloric acid. 30.2 g of catalyst (10% palladium on carbon wetted with 50% water, BASF type #286063) was added. A mixture of 95 vol. % N₂ and 5 vol. % H₂ (available from Linde Gas) was passed through the suspension for 6.5 hours at a flow rate of 80 L/h using a glass filter candle. The temperature was 20 to 25° C. and the overpressure was approx. 130 mbar. After HPLC had shown that the substrate had completely reacted, the catalyst was removed by filtration. The solution was concentrated in vacuo. 1.4 L water was given to the resultant liquid and the product was crystallized with the help of 110 mL 5% ammonia solution. The crystals were isolated using a Büchner funnel and dried at 35° C. in vacuo. 77.2 g (4S,4aS,5aR,12aS)-9-amino-4,7-bis(dimethylamino)-1,4,4a,5,5a,6,11,12a-octahydro-3,10,12,12a-tetrahydroxy-1,11-dioxo-2-naphthacencarboxamide hydrochloride dihydrate were obtained.

The purity of the product was 99.3% as determined using HPLC. The unreduced starting compound (4S,4aS,5aR,12aS)-4,7-bis(dimethylamino)-9-nitro-1,4,4a,5,5a,6,11,12a-octahydro-3,10,12,12a-tetrahydroxy-1,11-dioxo-2-naphthacencarboxamide runs at about 10.3 min in this assay and is barely detectable with a peak area of below 0.1%. For sake of comparability, the peak areas at 6.856, 7.072 and 7.663 are 0.14%, 0.18% and 0.11%, respectively. Thus reduction of the nitro compound to the corresponding amine was thus essentially complete.

1-14. (canceled)

15. A process for the reaction of a compound with hydrogen comprising:
reacting a compound with hydrogen using a hydrogen-containing gas comprising up to about 10 vol. % hydrogen and at least about 90 vol. % of an inert gas, wherein the compound to be reacted with hydrogen is provided in a liquid phase, and wherein the reaction is a hydrogenation reaction and the compound to be reacted with hydrogen contains a double bond or a triple bond selected from the group consisting of
NO₂ C═N C≡N C═O —N═N—.

16. The process according to claim 15, wherein the process further comprises providing the compound to be reacted with hydrogen as a liquid or dissolved, suspended or emulsified in the liquid phase.

17. The process according to claim 15, wherein the process further comprises passing the hydrogen-containing gas through the liquid phase.

18. The process according to claim 16, wherein the process further comprises passing the hydrogen-containing gas through the liquid phase.

19. The process according to claim 15, wherein the process further comprises conducting the reaction in the presence of a homogeneous or heterogeneous catalyst.

20. The process according to claim 16, wherein the process further comprises conducting the reaction in the presence of a homogeneous or heterogeneous catalyst.

21. The process according to claim 17, wherein the process further comprises conducting the reaction in the presence of a homogeneous or heterogeneous catalyst.

22. The process according to claim 19, wherein the process further comprises providing the catalyst comprising a platinum group metal or nickel.

23. The process according to claim 20, wherein the process further comprises providing the catalyst comprising a platinum group metal or nickel.

24. The process according to claim 21, wherein the process further comprises providing the catalyst comprising a platinum group metal or nickel.

25. The process according to claim 15, wherein the process further comprises providing nitrogen as the inert gas.

26. The process according to claim 16, wherein the process further comprises providing nitrogen as the inert gas.

27. The process according to claim 17, wherein the process further comprises providing nitrogen as the inert gas.

28. The process according to claim 19, wherein the process further comprises providing nitrogen as the inert gas.

29. The process according to claim 15, wherein the process further comprises providing the gas comprising about 0.1 vol. % to about 10 vol. % hydrogen and about 90 vol. % to about 99.9 vol. % of an inert gas.

30. The process according to claim 15, wherein the process further comprises providing the gas comprising about 1 vol. % to about 7 vol. % hydrogen and about 93 vol. % to about 99 vol. % of an inert gas.

31. The process according to claim 15, wherein the process further comprises providing the gas comprising about 2 vol. % to about 6 vol. % hydrogen and about 94 vol. % to about 98 vol. % of an inert gas.

32. The process according to claim 15, wherein the process further comprises providing the gas comprising about 5 vol. % hydrogen and about 95 vol. % of an inert gas.

33. A process for the reaction of a compound with hydrogen comprising:
reacting a compound with hydrogen using a hydrogen-containing gas comprising up to about 10 vol. % hydrogen and at least about 90 vol. % of an inert gas, wherein the compound to be reacted with hydrogen is provided in a liquid phase, wherein the reaction is a hydrogenolysis reaction.

34. The process according to claim 33, wherein the process further comprises providing the compound having a moiety selected from the group consisting of [chemical structure]
